SEQUENCE LISTING (1 sequence)

SEQ ID NO: 1 - protein, 9 aa, synthetic construct
FQWAVGHXL

(19) United States
(12) Patent Application Publication
(10) Pub. No.: US 2026/0335473 A1
(43) Pub. Date: Oct. 29, 2026

(54) NEW HETERODIMERS FOR PROSTATE AND BREAST CANCER

(71) Applicants: The Curators of the University of Missouri, Columbia, MO (US); The United States Government as represented by the Department of Veterans Affairs, Washington, DC (US)

(72) Inventors: Charles Jeffrey Smith, Columbia, MO (US); Carolyn J. Anderson, Columbia, MO (US); Michael R. Lewis, Columbia, MO (US)

(21) Appl. No.: 19/111,499

(22) PCT Filed: Sep. 13, 2023
(86) PCT No.: PCT/US2023/074095

§ 371 Date: Mar. 13, 2025

Related U.S. Application Data
(60) Provisional application No. 63/375,589, filed on Sep. 14, 2022.

(51) Int. Cl. A61K 51/08; A61P 35/00

(52) U.S. Cl. CPC A61K 51/088; A61P 35/00; A61K 2123/00

(57) ABSTRACT

Theranostic agents for targeting Gastrin Releasing Peptide Receptor (GRPR) and Prostate Specific Membrane Antigen (PSMA) expressing cells, and use of these agents for detection, staging, and therapy of primary and metastatic prostate cancer (PCa) are disclosed.

### CROSS REFERENCE TO RELATED APPLICATIONS

This application claims priority to U.S. Provisional Application No. 63/375,589 filed on Sep. 14, 2022, which is hereby incorporated by reference in its entirety.

### STATEMENT OF GOVERNMENT SUPPORT

This invention was made with government support under 1I01BX003392 awarded by the U.S. Department of Veterans' Affairs and U01 HL152410 awarded by the National Institutes of Health (NIH). The government has certain rights in the invention.

### INCORPORATION OF SEQUENCE LISTING

A paper copy of the Sequence Listing and a computer readable form of the Sequence Listing containing the file named “22UMC036PCT”, which is 1899 bytes in size (as measured in MICROSOFT WINDOWS® EXPLORER), are provided herein and are herein incorporated by reference. This Sequence Listing consists of SEQ ID No:1.

### BACKGROUND OF THE DISCLOSURE

The present disclosure relates generally to the development of radiotracers for targeting Gastrin Releasing Peptide Receptor (GRPR) and Prostate Specific Membrane Antigen (PSMA) expressing cells.

Prostate cancer is the second most leading cause of cancer deaths in men in the U.S. with a projected death total of 34,500 in 2022. While the 5-year survival rate is near 100% for patients presenting with locoregional disease, the death rate increases dramatically (29%) for patients presenting with metastatic disease.

Most existing therapies, such as chemotherapy and external beam radiation, are nonspecific, resulting in collateral damage to healthy tissues and dose-limiting side effects. Therefore, patients with metastatic prostate cancer might respond better to alternative treatments, such as targeted radiotherapy with radiolabeled antibodies, peptides, or small molecules. However, targeted radiotherapy, ideally, should also be accompanied by a targeted radioimaging correlate for γ scintigraphy, single-photon emission computed tomography (SPECT), or positron emission tomography (PET).

Prostate specific antigen (PSA) is the most common biomarker tool used clinically for early disease detection with relatively high precision. However, in a number of cases, the PSA determination results in false positive outcomes leading to ambiguity in clinical decision making. With the evolution of receptor-based targeting, there has been a resurgence in development of molecular markers, which has led to high end detection of the disease at various stages of tumor growth enabling effective clinical management of the disease.

It has been reported that Gastrin Releasing Peptide (GRP) receptor subtype 2 is overexpressed on several types of human cancer cells with an expression rate of approximately 84% on primary prostate tumors; lymph node and bone metastases are 52.9% and 85.7%, respectively. It has been shown previously that peptide-based agonist/antagonist radioligands are clinically useful for molecular imagining and therapy.

Additionally, prostate specific membrane antigen (PSMA), also known as folate hydrolase 1 is present on the neovasculature of most solid tumors, with 95% of all prostate cancers expressing PSMA. Lymph node and bone metastases are 100% in PSMA expression. Previously, tumor targeting by radiolabeled urea-based PSMA inhibitors has shown to be clinically useful for molecular imaging.

Based on the foregoing, there is a significant need for a single agent for detection, staging and therapy. The present disclosure provides for novel radiotracers that will be able to reach a larger cohort of patients with prostate cancer. Further, similar to prostate cancer, there are receptors for PSMA and GRPR on breast cancer tissues. Accordingly, these radiotracers may be useful in patients with breast cancer as well.

### BRIEF DESCRIPTION OF THE DISCLOSURE

The present disclosure is generally directed to radiotracer agents for targeting Gastrin Releasing Peptide Receptor (GRPR) and Prostate Specific Membrane Antigen (PSMA) expressing cells, and use of these agents for detection, staging, and therapy of primary and metastatic prostate cancer (PCa). As there are receptors for PSMA and GRPR on breast cancer tissues. Accordingly, these radiotracers may be useful in patients with breast cancer as well

In one aspect, the present disclosure relates to a radiotracer compound having the formula of Formula (I):

DUPA-X-M-Y-X-BBN ANT Formula (I)
wherein:
DUPA is (2-[3-(1,3-dicarboxypropyl)-ureido]pentanedioic acid);
X are independently selected from the group consisting of 4-amino-1-carboxymethly-piperidine (CP), 5-ammoniumvaleric acid (5-Ava), 6-aminohexanoic acid (6-Ahx), 8-aminooctanoic acid (8-AOC), and paraaminobenzoic acid (AMBA);
M is selected from the group consisting of: [⁶⁸Ga], [¹⁷⁷Lu], [⁶⁴Cu], [⁶⁷Cu] and [¹¹¹IN];
Y is a metal complexing agent selected from the group consisting of: a dodecane tetraacetic acid (DOTA), [1,4,7-triazacyclododecane-1,4,7-triacetic acid](NOTA), and cross-bridged TETA phosphonic acid (CB-TE1A1P); and
BBN ANT is a BBN antagonist ligand of SEQ ID NO:1.

In another aspect, the present disclosure relates to a method for detecting prostate cancer (PCa), the method comprising administering the radiotracer compound of any one of claims 1-18 to a subject suspected to be at risk of PCa.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts an exemplary structure of a compound of Formula (IA).

FIG. 2 depicts exemplary structures of compounds of Formula (IB), particularly wherein X is 8-aminooctanoic acid (8-AOC) or 4-amino-1-carboxymethly-piperidine (CP).

FIG. 3 depicts the scheme for the synthesis of Heterodimer peptide-CB cyclam chelator conjugates as prepared and analyzed in the Examples.

FIGS. 4A-4D depict depicts HPLC (radioactive and UV) profiles of [DUPA-6-Ahx-([^{64/67}Cu]-NODAGA)-8AOC/CP-BBN ANT] as analyzed in Example 1.

FIGS. 5A-5D depict concentration of [DUPA-6-Ahx-([^{64/67}Cu]-NODAGA)-8AOC/CP-BBN ANT] as analyzed in Example 1.

FIGS. 6A-6F depict stability of [DUPA-6-Ahx-([^{64/67}Cu]-NODAGA)-8AOC/CP-BBN ANT] as analyzed in Example 1.

FIGS. 7A & 7B depict in vivo analysis of [DUPA-6-Ahx-([^{64/67}Cu]-NODAGA)-8AOC/CP-BBN ANT] in the GRPR-expressing tumors, PC3 (FIG. 7A) and PC3 PIP (FIG. 7B).

FIGS. 8A & 8B depict in vivo analysis of (FIG. 8A) [DUPA-6-Ahx-([^{64/67}Cu]-NODAGA)-8AOC-BBN ANT] and (FIG. 8B) [DUPA-6-Ahx-([64/67Cu]-NODAGA)-CP-BBN ANT] in GRPR-expressing tumors.

FIGS. 9A & 9B depict in vivo analysis of (FIG. 9A) [DUPA-6-Ahx-([^{64/67}Cu]-NODAGA)-8AOC-BBN ANT] and (FIG. 9B) [DUPA-6-Ahx-([64/67Cu]-NODAGA)-CP-BBN ANT] in PSMA-expressing tumors.

FIGS. 10A-10D depict HPLC (radioactive and UV) profiles of ⁶⁴Cu/^{nat}Cu labeled Heterodimer peptide-CB cyclam conjugates.

FIGS. 11A-11D depict stability of [DUPA-6-Ahx-([^{64/67}Cu]-NODAGA)-8AOC/CP-BBN ANT] as analyzed in Example 2.

FIGS. 12A & 12B depict biodistribution data of (FIG. 12A)⁶⁴Cu-DUPA-6-Ahx-(Lys-TE1A)-8AOC-BBN-ANT and (FIG. 12B)⁶⁴Cu-DUPA-6-Ahx-(Lys-TE1P)-8AOC-BBN-ANT in Nude mice bearing PC3-PIP tumors as analyzed in Example 2.

FIGS. 13A-13D depicts biodistribution data of (FIG. 13A)⁶⁴Cu-DUPA-6-Ahx-(Lys-TE1A)-8AOC-BBN-ANT and (FIG. 13C)⁶⁴Cu-DUPA-6-Ahx-(Lys-TE1P)-8AOC-BBN-ANT and critical ratios of (FIG. 13B)⁶⁴Cu-DUPA-6-Ahx-(Lys-TE1A)-8AOC-BBN-ANT and (FIG. 13D)⁶⁴Cu-DUPA-6-Ahx-(Lys-TE1P)-8AOC-BBN-ANT in Nude mice bearing PC3 tumors as analyzed in Example 2.

FIGS. 14A & 14B depict critical ratios of (FIG. 14A)⁶⁴Cu-DUPA-6-Ahx-(Lys-TE1A)-8AOC-BBN-ANT and (FIG. 14B)⁶⁴Cu-DUPA-6-Ahx-(Lys-TE1P)-8AOC-BBN-ANT in Nude mice bearing PC3-PIP tumors as analyzed in Example 2.

### DETAILED DESCRIPTION

Unless defined otherwise, all technical and scientific terms used herein have the same meaning as commonly understood by one of ordinary skill in the art to which the disclosure belongs.

The present disclosure relates to theranostic agents for targeting Gastrin Releasing Peptide Receptor (GRPR) and Prostate Specific Membrane Antigen (PSMA) expressing cells, and use of these agents for detection, staging, and therapy of primary and metastatic prostate cancer (PCa). These new radiotracers would allow the nuclear medicine physician to capture all stages of the disease, a requirement which could be severely limited by monovalent GRPR- or PSMA-targeting ligands, where one or the other molecular biomarkers is expressed in low numbers or not at all.

Generally, the radiotracer compounds of the present disclosure will have the formula of Formula (I):

DUPA-X-M-Y-X-BBN ANT Formula (I)
wherein:
DUPA is (2-[3-(1,3-dicarboxypropyl)-ureido]pentanedioic acid), a small molecule PSMA-targeting probe;
X are independently selected from the group consisting of 4-amino-1-carboxymethly-piperidine (CP), 5-ammoniumvaleric acid (5-Ava), 6-aminohexanoic acid (6-Ahx), 8-aminooctanoic acid (8-AOC), and paraaminobenzoic acid (AMBA);
M is selected from the group consisting of: [⁶⁸Ga], [¹⁷⁷Lu], [⁶⁴Cu], [⁶⁷Cu], and [¹¹¹IN];
Y is a metal complexing agent selected from the group consisting of: a dodecane tetraacetic acid (DOTA, and particularly, [1,4,7,10-tetraazacyclododecane-1,4,7,10-tetraacetic acid](DO3A), [1,4,7-triazacyclododecane-1,4,7-triacetic acid](NOTA), and particularly its derivative, NODAGA, and cross-bridged TETA phosphonic acid (CB-TE1A1P); and
BBN ANT is a BBN antagonist ligand of SEQ ID NO:1 [D-Phe-Gln-Trp-Ala-Val-Gly-His-Sta-Leu-NH₂].

In some suitable embodiments, the theranostic agents include [⁶⁸Ga], [¹⁷⁷Lu] and/or [¹¹¹IN] (Table 1), two of which, [⁶⁸Ga] and [¹⁷⁷Lu], are well-validated and clinically used diagnostic and therapeutic radioisotopes. [⁶⁸Ga] is a generator-produced ([⁶⁸Ge]/[⁶⁸Ga]) radionuclide with a half-life of 1.13 h. The physical half-life of [⁶⁸Ga] is sufficiently long enough for radiopharmaceutical preparation, quality control validation, and PET molecular imaging investigations. [⁶⁸Ga] decays 89% through positron emission (maximum energy of 1.899 MeV, average energy of 0.89 MeV). [Ga³⁺] is considered to be a hard metal center, preferentially complexing to hard donor atoms such as oxygen and nitrogen.

[¹⁷⁷Lu] is a rare-earth radionuclide that is produced in moderate specific activity (740 GBq/mg) by direct neutron capture of enriched [¹⁷⁶Lu] via the (¹⁷⁶Lu(n,γ)¹⁷⁷Lu) nuclear reaction. [¹⁷⁷Lu] has a half-life of 6.71 d, decays by beta emission (E_{βmax}=0.497 MeV), and emits two SPECT imaging gamma photons (113 keV, 3% and 208 keV, 11%). In addition, high specific activity, no-carrier-added ¹⁷⁷Lu can also be prepared by an indirect neutron capture nuclear reaction on a ¹⁷⁶Yb target. Like [Ga³⁺], [Lu³⁺] is also considered to be a hard metal center, preferentially complexing to hard donor atoms such as oxygen and nitrogen.

TABLE 1
Description of In Vitro and In Vivo Assays to Validate the Cell-targeting
Capacity of Agents in GRPR/PSMA-expressing Cells

| | Physical | | |
|---|---|---|---|
| Isotope | Characteristics | Availability | Specific Activity |
| Gallium-68 | t_{1/2} = 1.13 h, β⁺, 1.899 | Limited, Ge-68/ | High |
| | MeV | Ga-68 | |
| | | Generator | |
| Indium-111 | t_{1/2} = 2.80 d, γ, 171 | Widely Available, | High |
| | and 245 keV | Cyclotron | |
| Copper-64 | t_{1/2} = 12.7 h, β⁺, 0.65 | Produced | High |
| | MeV; β⁻, 0.57 MeV | | |
| Lutetium- | t_{1/2} = 6.71 d, γ, 208 | Widely Available, | Low (High, |
| 177 | keV; β⁻, 0.497 MeV | Cyclotron | Indirect Neutron |
| | | | Capture) |
| Copper-67 | t_{1/2} = 2.58 d, γ, 93 and | Produced | High |
| | 185 keV; β⁻, 0.562 | | |
| | MeV | | |

Particularly, in these embodiments, the present disclosure is generally directed to the development of a new generation of theranostic, heterobivalent radioligands of formula (IA):

DUPA-6-Ahx-([M]-DOTA)-X-BBN ANT Formula (IA)
wherein
M=[⁶⁸Ga] or [¹⁷⁷Lu] or [¹¹¹IN];
DOTA=dodecane tetraacetic acid metal complexing agent, particularly (DO3A)=[1,4,7,10-tetraazacyclododecane-1,4,7,10-tetraacetic acid];
6-Ahx=6-aminohexanoic acid;
X is selected from the group consisting of 4-amino-1-carboxymethly-piperidine (CP), 5-ammoniumvaleric acid (5-Ava), 6-aminohexanoic acid (6-Ahx), 8-aminooctanoic acid (8-AOC), or paraaminobenzoic acid (AMBA), and suitably, is CP;
BBN ANT=BBN antagonist ligand of SEQ ID NO:1; and
DUPA=(2-[3-(1,3-dicarboxypropyl)-ureido]pentanedioic acid), a small molecule PSMA-targeting probe

Suitable radioligands are [DUPA-6-Ahx-([⁶⁸Ga] Ga-DO3A)-CP-BBN ANT] and [DUPA-6-Ahx-([¹⁷⁷Lu]Lu-DO3A)-CP-BBN ANT] radioligands. These new radiotracers differ from previously produced radioligands by introduction of 4-amino-1-carboxymethly-piperidine as a new pharmacokinetic modifier as in the structure of clinically-used RM2 (Minamimoto R et al. J Nucl Med 2016; 57:557-62). It has been shown that radioligands of this type have high selectivity and affinity for both GRPR and PSMA (Bandari R P et al., Journal of Medicinal Chemistry, 2021).

In other suitable embodiments, copper can be used as the radioisotope. [⁶⁴Cu] is a cyclotron-produced radionuclide with a sufficiently long-enough half-life (12.7 h) to be considered readily available for radiopharmaceutical preparation, quality control, drug incorporation, circulation, and patient imaging. [⁶⁴Cu] is produced by irradiation of [⁶⁴Ni] via the (⁶⁴Ni(p,n)⁶⁴Cu) nuclear reaction. [⁶⁴Cu]Cu-labelled radiopharmaceuticals are of primary interest due to the ideal nuclear characteristics of [⁶⁴Cu]: [⁶⁴Cu](t1/2=12.7 h; Eβ⁺max=0.65 MeV (17.5%); Eβ⁻max=0.57 MeV (38.5%); electron capture (EC) (44.0%)).

Interest in [⁶⁷Cu] has recently been reinvigorated by the breakthrough in production of significant quantities of high specific activity radiometal. [⁶⁷Cu] is produced in high specific activity (5.55 GBq/μg) by irradiation of a highly enriched [⁶⁸Zn] target via the (⁶⁸Zn(γ,p)⁶⁷Cu) nuclear reaction. [⁶⁷Cu] has a half-life of 2.58 d, decays by beta emission (Eβ⁻max=0.562 MeV) and emits two SPECT imaging gamma photons (93 keV, 16% and 185 keV, 49%).

Copper (II) is considered to be a hard metal center, preferentially complexing to hard donor atoms such as oxygen and nitrogen. The Anderson research group, in collaboration with Wong and Weisman at the University of New Hampshire, developed cross-bridged macrocyclic chelators that have extraordinary kinetic stability with Cu(II) (Boswell C A et al., J Med Chem 2004; 47:1465-74). Since that time, they have shown that phosphonate-based moieties also bind stably to Cu(II). The cross-bridged chelator with one phosphonate pendant arm for coordination of Cu(II) and one carboxylate arm for conjugation to peptide-based biomolecules (CB-TE1A1P), allows for complexation of copper radionuclides under mild conditions while maintaining in vivo stability.

In some embodiments, usage of (poly)aminocarboxylate complexing agents based upon NOTA [1,4,7-triazacyclododecane-1,4,7-triacetic acid] to complex [⁶⁸Ga], [⁶⁴Cu], and [⁶⁷Cu] are disclosed. Compounds of these embodiments have the formula of formula (IB):

DUPA-6-Ahx-([M]-NOTA)-X-BBN ANT Formula (IB)
wherein
M is selected from the group consisting of [⁶⁸Ga], [⁶⁴Cu], and [⁶⁷Cu]; and
X is selected from the group consisting of 4-amino-1-carboxymethly-piperidine (CP), 5-ammoniumvaleric acid (5-Ava), 6-aminohexanoic acid (6-Ahx), 8-aminooctanoic acid (8-AOC), or paraaminobenzoic acid (AMBA), and suitably, is CP.

In other of the present embodiments, the heterobivalent PSMA-/GRPR-targeting agents are conjugated with CBTE1A1P for radiolabeling with [⁶⁴Cu] and [⁶⁷Cu] and have the formula of formula (IC):

DUPA-6-Ahx-([M]-CB-TE1A1P)-X-BBN ANT Formula (IC)
wherein
M is [⁶⁴Cu] or [⁶⁷Cu]; and
X is selected from the group consisting of 4-amino-1-carboxymethly-piperidine (CP), 5-ammoniumvaleric acid (5-Ava), 6-aminohexanoic acid (6-Ahx), 8-aminooctanoic acid (8-AOC), or paraaminobenzoic acid (AMBA), and suitably, is CP.

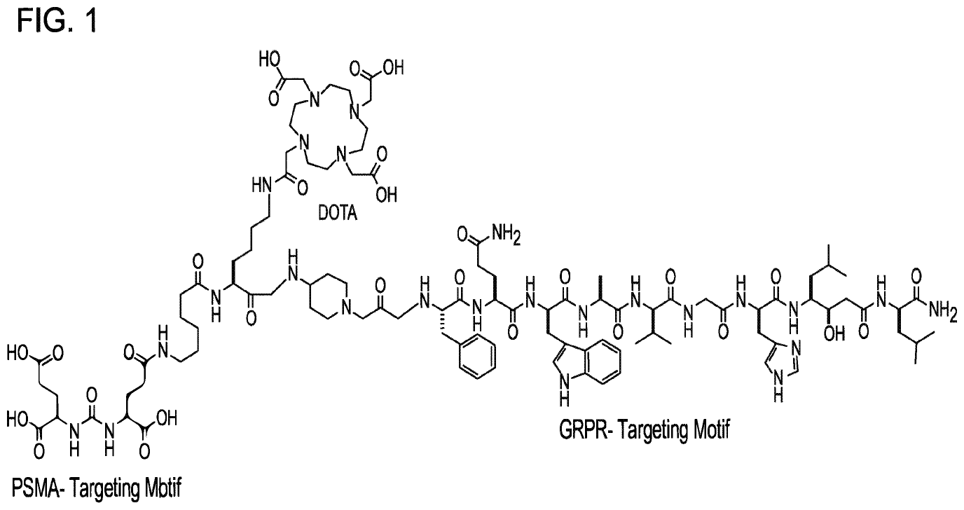

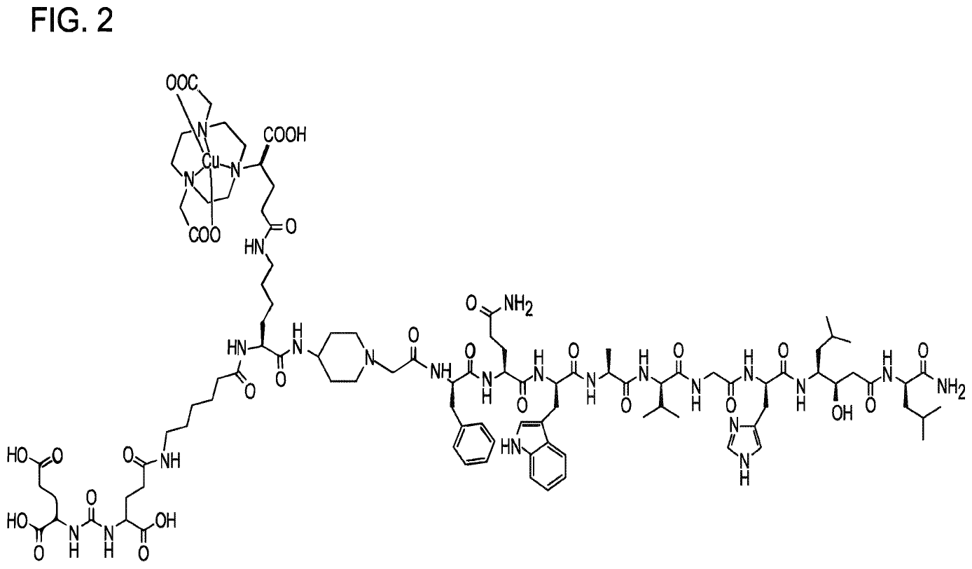

Suitable new theranostic, heterobivalent radioligands having either formula (IB) or (IC) include: [DUPA-6-Ahx-([^{64/67}Cu]Cu-NOTA)-CP-BBN ANT], [DUPA-6-Ahx-([^{64/67}Cu]Cu-NOTA)-8-Aoc-BBN ANT], [DUPA-6-Ahx-([⁶⁸Ga] Ga-NOTA)-CP-BBN ANT], [DUPA-6-Ahx-([⁶⁸Ga] Ga-NOTA)-8-Aoc-BBN ANT], [DUPA-6-Ahx-([^{64/67}Cu]Cu-CB-TE1A1P)-CP-BBN ANT], and [DUPA-6-Ahx-([^{64/67}Cu]Cu-CB-TE1A1P)-8-Aoc-BBN ANT], FIGS. 1-3). In the context of the structural motifs, M=[⁶⁸Ga], [⁶⁴Cu], or [⁶⁷Cu]; 6-Ahx=6-aminohexanoic acid; 8-Aoc=8-aminooctanoic acid, CP=4-amino-1-carboxymethlypiperidine; NOTA (NOTA/NODAGA)=[1,4,7-triazacyclododecane-1,4,7-triacetic acid] copper complexing agents; CB-TE1A1P=cross-bridged TETA phosphonic acid, a cyclam-based (phosphonate), cross-bridged copper complexing agent; BBN ANT=BBN antagonist ligand [D-Phe-Gln-Trp-Ala-Val-Gly-His-Sta-Leu-NH₂](SEQ ID NO:1), a GRPR-specific peptide targeting probe; and DUPA=(2-[3-(1,3-dicarboxypropyl)-ureido]pentanedioic acid), a small molecule PSMA-targeting probe. For derivatives containing NODAGA, the use of [⁶⁸Ga] radiometal for PET molecular imaging is disclosed.

The present disclosure is further directed to use of the radiotracer compounds for detecting prostate cancer (PCa) or breast cancer. Suitably, in one particular embodiment, the radiotracer compounds are used for detecting prostate cancer, and particularly, metastatic prostate cancer. Generally, methods of using the radiotracer compounds include administering the radiotracer compounds described herein to a subject at risk of prostate cancer or breast cancer.

As defined herein, “a subject at risk” refers to a subset of individuals who are susceptible to, or at elevated risk of, having pancreatic or breast cancer. A subject can be susceptible to, or at elevated risk of, experiencing symptoms due to family history, age, environment, and/or lifestyle. Based on the foregoing, because some of the methods embodiments of the present disclosure are directed to specific subsets or subclasses of identified individuals (that is, the subset or subclass of subjects having or suspected of having pancreatic or breast cancer), not all individuals will fall within the subset or subclass of individuals as described herein for certain diseases, disorders or conditions. The term “subject” includes both human subjects and animal subjects. Particularly suitable human subjects include pediatric human subjects, adolescent human subjects and adult human subjects. As used herein, “pediatric human subject” refers to a human subject ranging in age from about 2 years old to about 9 years old. As used herein, “adolescent human subject” refers to a human subject having an age of about 10 years old to about 19 years old. As used herein, “adult human subject” refers to a human subject having an age of 19 and older.

As used herein, “susceptible” and “at risk” refer to having little resistance to a certain disease, disorder or condition, including being genetically predisposed, having a family history of, and/or having symptoms of the disease, disorder or condition.

Generally, positron Emission Tomography (PET) could be used to assess the targeting capabilities of the radiotracer compounds in GRPR-/PSMA-expressing tumors. MicroCT and Ti- and diffusion-weighted microMRI images could be used to assist in anatomically correlating regions of physiological accumulation and retention of tracer compounds determined by PET in order to identify primary or metastatic pancreatic or breast cancer.

Once detected, the methods of the present disclosure could further include treating the subject found to have pancreatic or breast cancer. As used herein, “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders associated with administration of the therapy. Treatments as known in the art of pancreatic or breast cancers (e.g., surgery, radiation, chemotherapy or a combination of these) could be administered to the subject.

### EXAMPLES

### Example 1

In this Example, heterobivalent radiotracers [DUPA-6-Ahx-([M]-NODAGA)-8-Aoc-BBN ANT] and [DUPA-6-Ahx-([M]-NODAGA)-CP-BBN ANT] were prepared and analyzed for their potential for usage as a single agent with the true matched-pair ⁶⁴Cu/⁶⁷Cu isotopes for therapeutic and diagnostic use and positron emitting ⁶⁸Ga diagnostic isotope.

### General Materials

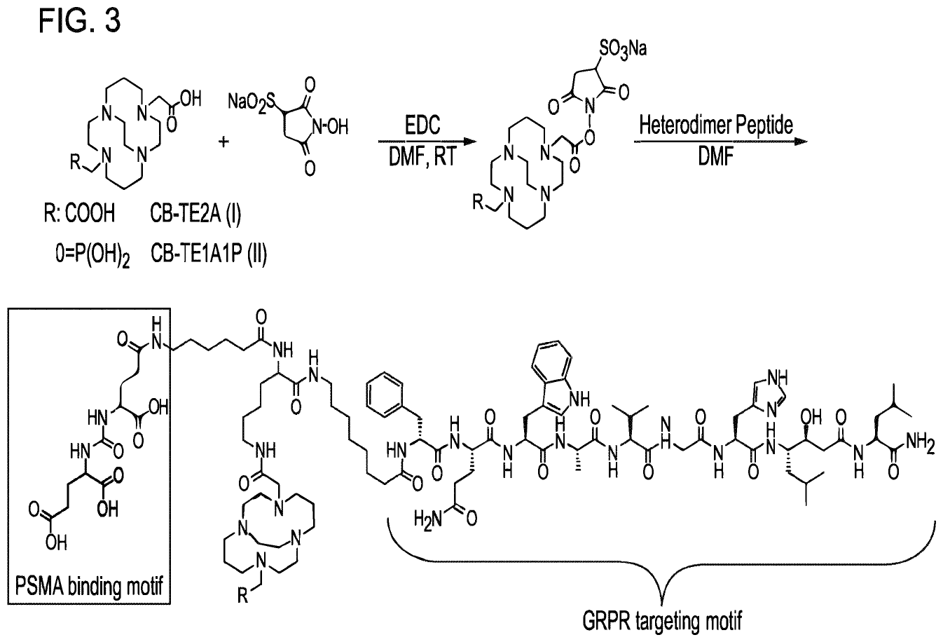

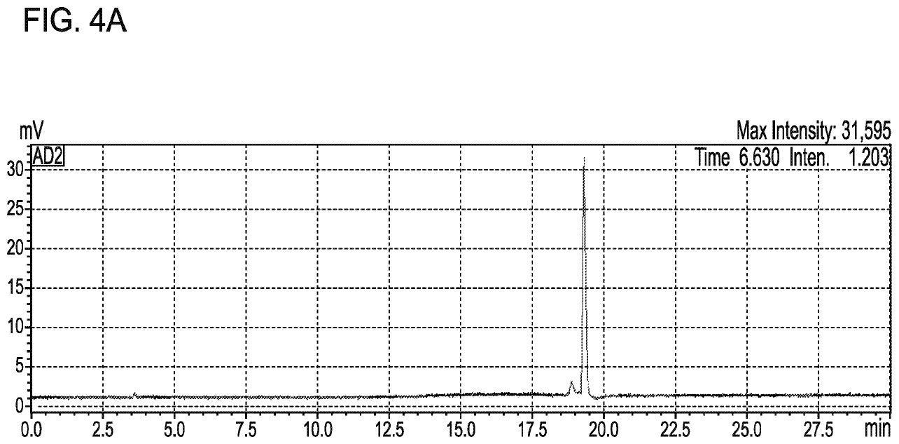

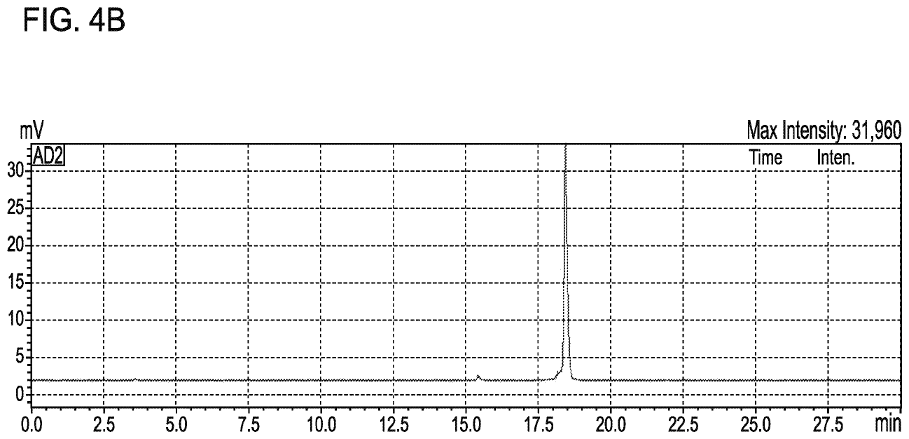

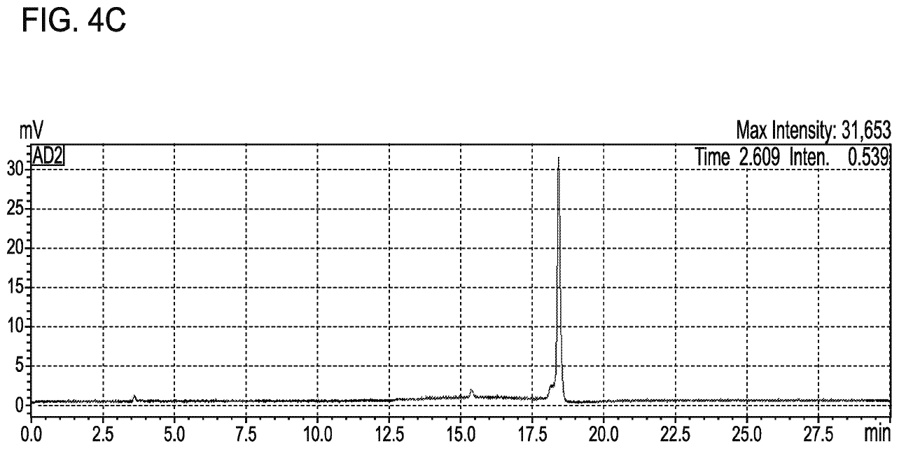

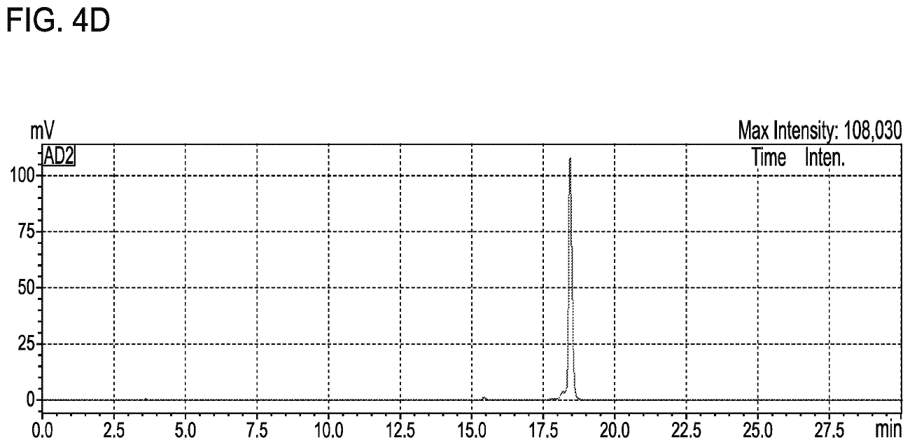

GRPR/PSMA targeting ligands [DUPA-6-Ahx-([DO3A)-CP-BBN ANT], [DUPA-6-Ahx-([DO3A)-8-Aoc-BBN ANT], [DUPA-6-Ahx-(NODAGA)-8-Aoc-BBN ANT], and [DUPA-6-Ahx-(NODAGA)-CP-BBN ANT](FIGS. 1 & 2) were purchased from EZBiolab (Carmel, IN, USA). [DUPA-6-Ahx-(CB-TE1A/TE1P)-8-Aoc-BBN ANT] and [DUPA-6-Ahx-(CB-TE1A/TE1P)-CP-BBN ANT](FIG. 3) were prepared and characterized by liquid electrospray ionization-mass spectrometry (ESI-MS), and used without further purification. Natural metals Lutetium(III) Chloride, Gallium(III) Chloride, and Copper(II) Chloride hydrate were purchased from Acros Organics and Sigma Aldrich. Blocking agents Bombesin 1-14 and 2-(Phosphonomethyl)pentanedioic acid were purchased from Sigma-Aldrich Chemical Company (St. Louis, MO, USA) and Tocris Bioscience (Bristol, UK). All other solvents and reagents were purchased from Fisher Scientific (Pittsburgh, PA, USA) or Sigma-Aldrich Chemical Company (St. Louis, MO, USA). The PC-3 human prostate carcinoma cell lines were purchased from American Type Tissue Culture Center (ATCC, Rockland, MD) and the PC3-PIP cell lines received from Professor Martin Pomper (John Hopkins University). The cells were maintained in 45% RPMI 1640, 45% Ham's F-12, and 10% heat-inactivated Fetal Bovine Serum (FBS). All metallated peptide conjugates were purified via RP-HPLC (reversed phase-high performance liquid chromatography) performed on an SCL-10A Shimadzu system (Kyoto, Japan) with an analytical Phenomenex Jupiter Proteo C-18 column. The HPLC chromatographic profiles of [DUPA-6-Ahx-([DO3A)-CP-BBN ANT], [DUPA-6-Ahx-([DO3A)-8-Aoc-BBN ANT], [DUPA-6-Ahx-(NODAGA)-8-Aoc-BBN ANT], and [DUPA-6-Ahx-(NODAGA)-CP-BBN ANT] are shown in FIGS. 4A-4D.

Preparation and Characterization of ^{nat}Metallated Complexes

[DUPA-6-Ahx-([DO3A)-CP-BBN ANT], [DUPA-6-Ahx-([DO3A)-8-Aoc-BBN ANT], [DUPA-6-Ahx-(NODAGA)-8-Aoc-BBN ANT], [DUPA-6-Ahx-(NODAGA)-CP-BBN ANT], [DUPA-6-Ahx-(CB-TE1A/TE1P)-8-Aoc-BBN ANT], and [DUPA-6-Ahx-(CB-TE1A/TE1P)-CP-BBN ANT] were metalated on the macroscopic level by addition of 1.5 mol of the chloride salts of either ^{nat}Cu/^{nat}Ga/^{nat}Lu to 1 mol of the heterodimer (100 μL 0.4 M NH₄OAc, 300 μL 0.05M HCl pH 4.0) with heating (80°, 45 min). Metalated peptides were characterized by ESI-MS.

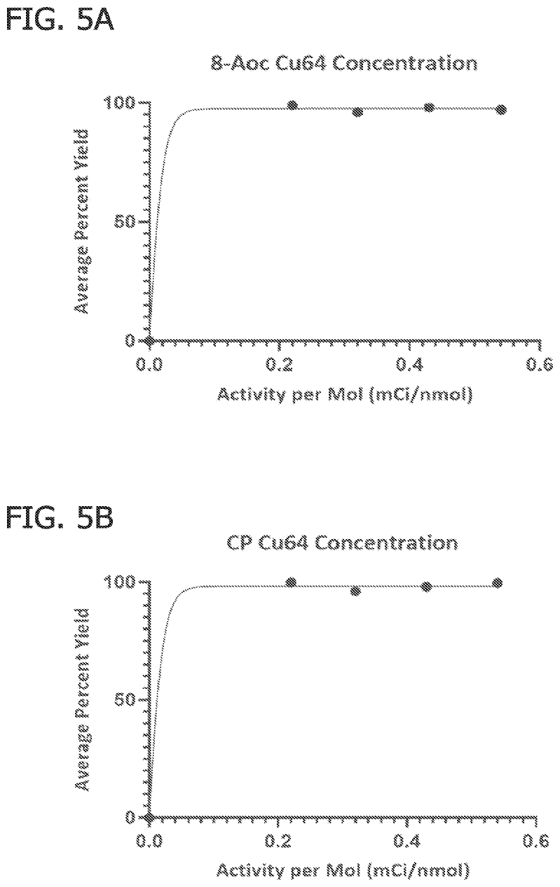

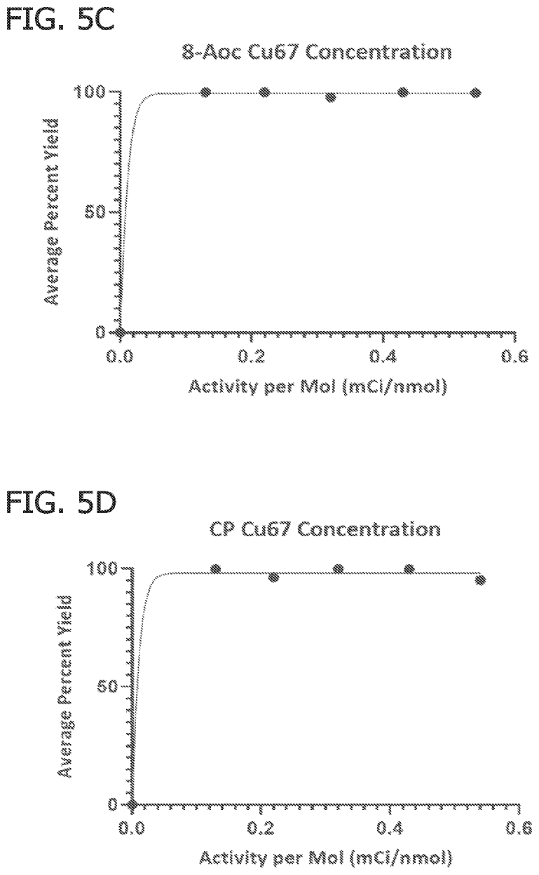

### Preparation of Radiolabeled Complexes

[DUPA-6-Ahx-([DO3A)-CP-BBN ANT], [DUPA-6-Ahx-([DO3A)-8-Aoc-BBN ANT], [DUPA-6-Ahx-(NODAGA)-8-Aoc-BBN ANT], [DUPA-6-Ahx-(NODAGA)-CP-BBN ANT], [DUPA-6-Ahx-(CB-TE1A/TE1P)-8-Aoc-BBN ANT], and [DUPA-6-Ahx-(CB-TE1A/TE1P)-CP-BBN ANT] were radiolabeled by addition of 500 mCi of either ¹⁷⁷LuCl₃, ⁶⁸GaCl₃, or ^{64/67}CuCl₂ to 1 nmol of peptide (100 μL of 0.4 M NH₄OAc) with heating at 80° for 45 minutes. 50 μL of 10 mM DTPA was added to scavenge any unbound metal and radiochemical purity was determined via RP-HPLC. All complexes were radiolabeled in very high radiochemical purity (>95% radiochemical purity) and isolated by C-18 sep-pak prior to all other investigations (see FIGS. 5A-5D).

### Stability Investigations of Radiolabeled Complexes

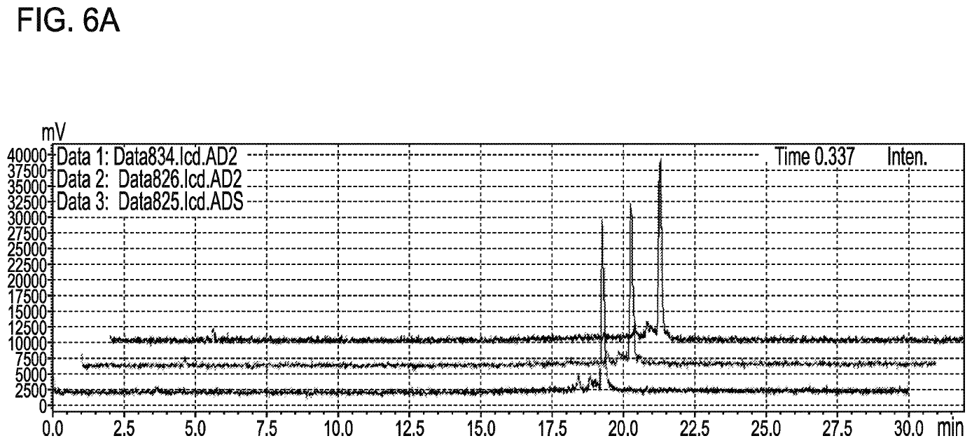

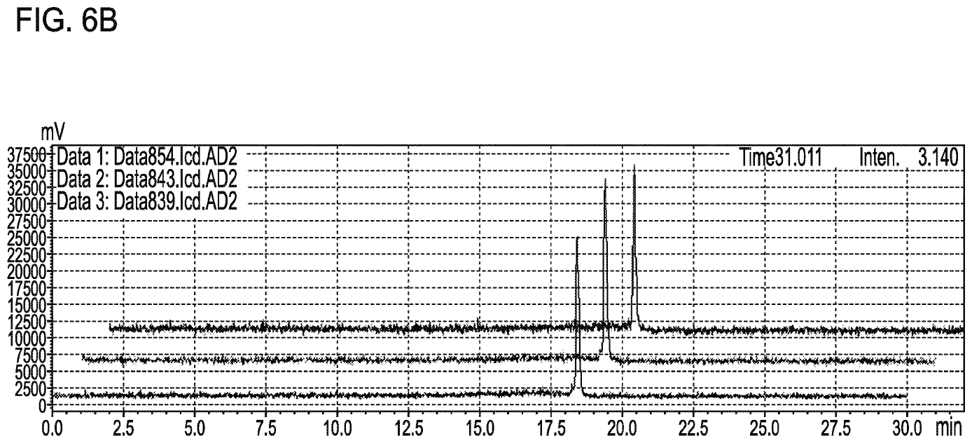

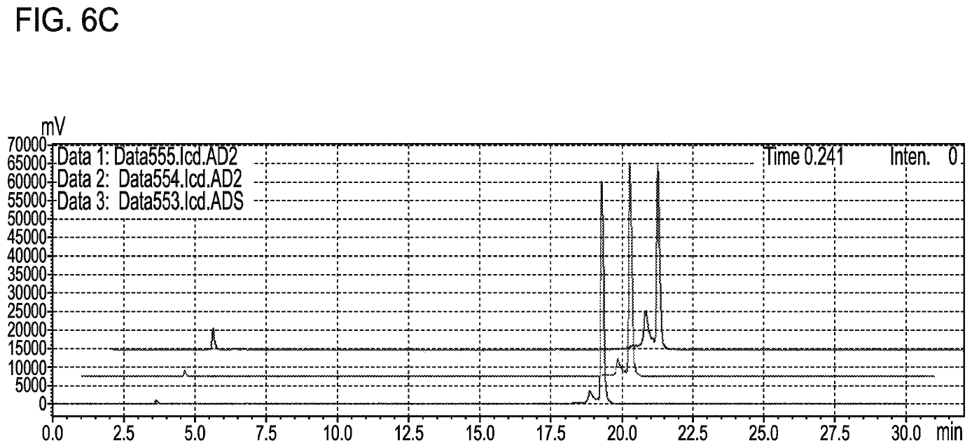

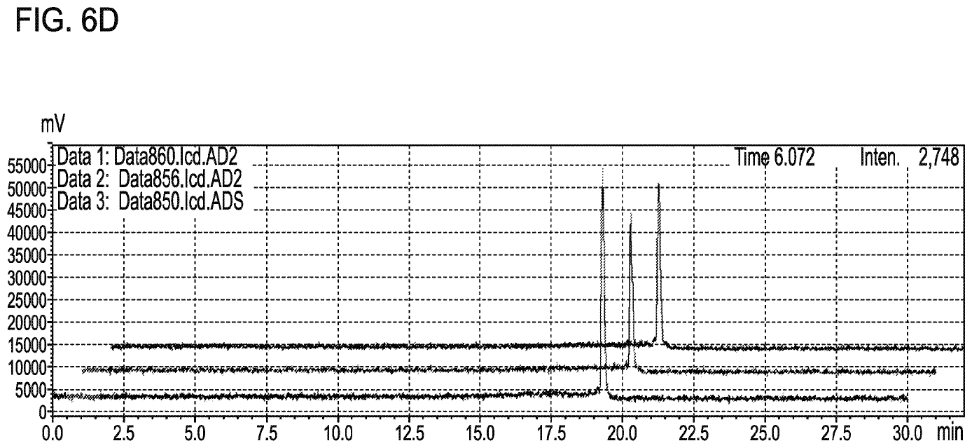

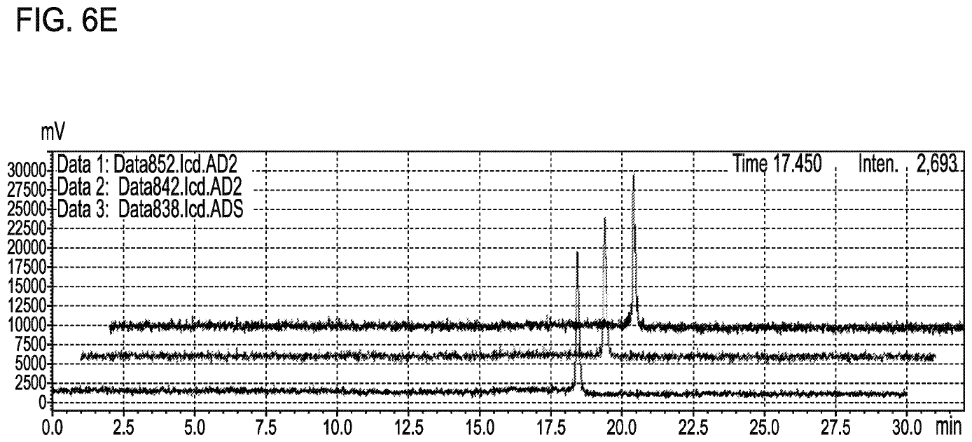

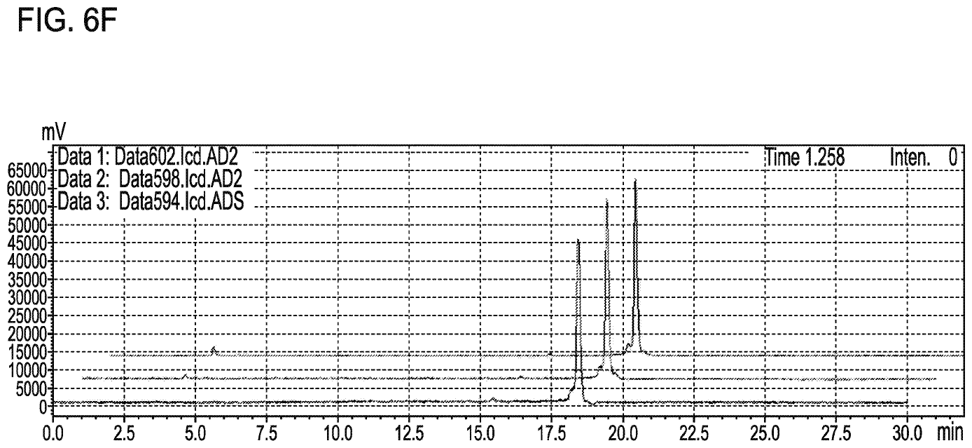

Stability studies of the radiotracers were completed in phosphate buffered saline (PBS) and human serum (HS) followed by RP-HPLC analyses at different time points. PBS studies were determined by incubation of the radioligand of interest in 500 μL of PBS at room temperature. For serum stability, 500 μL of human serum was incubated with the radioligand at 37 °C. After incubation at the selected time points, 500 μL of acetonitrile was added to denature the protein before placing in a centrifuge for 5 mins at 7000 rpm to form a protein pellet. The supernatant was removed from the pellet and each of the fractions were counted in a dose calibrator. Each radiotracer was also evaluated for stability via RP-HPLC (see FIGS. 6A-6F).

### In Vivo: Biodistribution and Pharmacokinetics in Tumor Bearing Mice

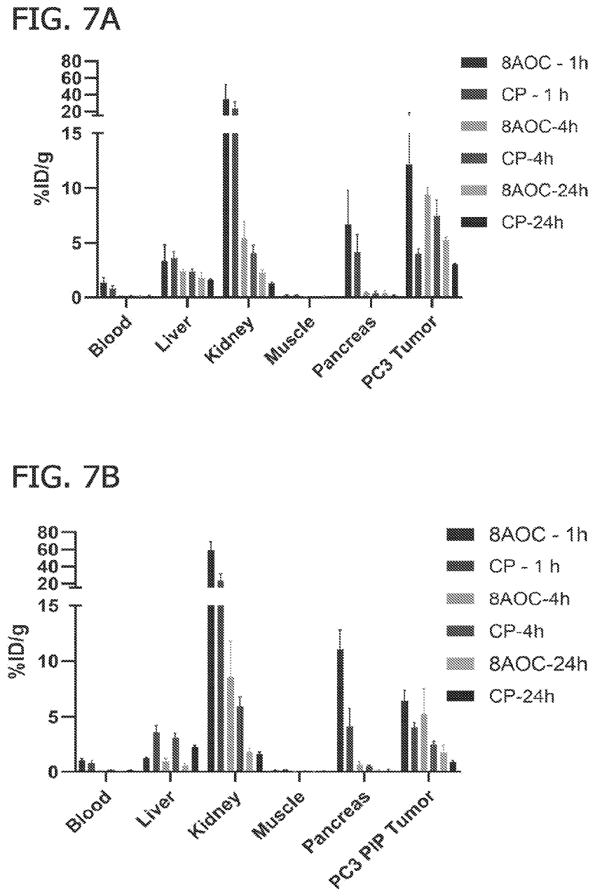

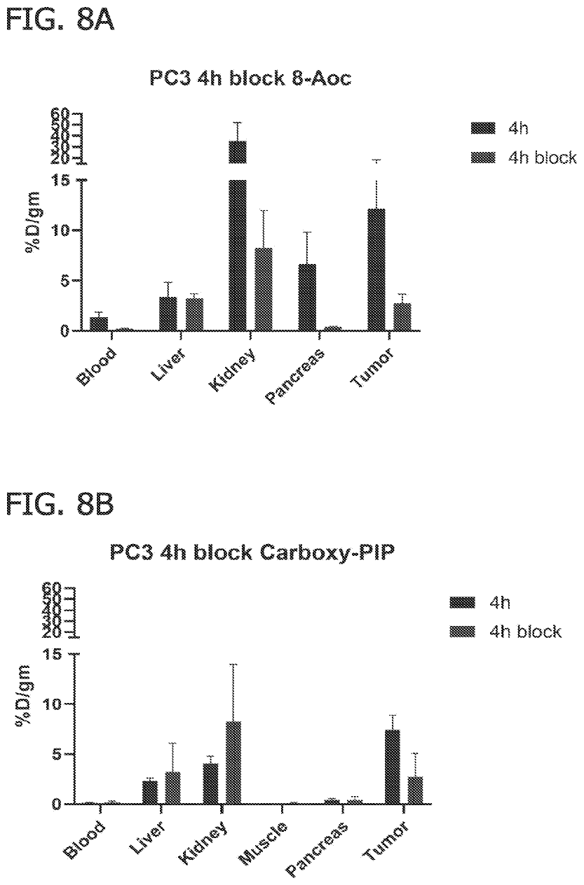

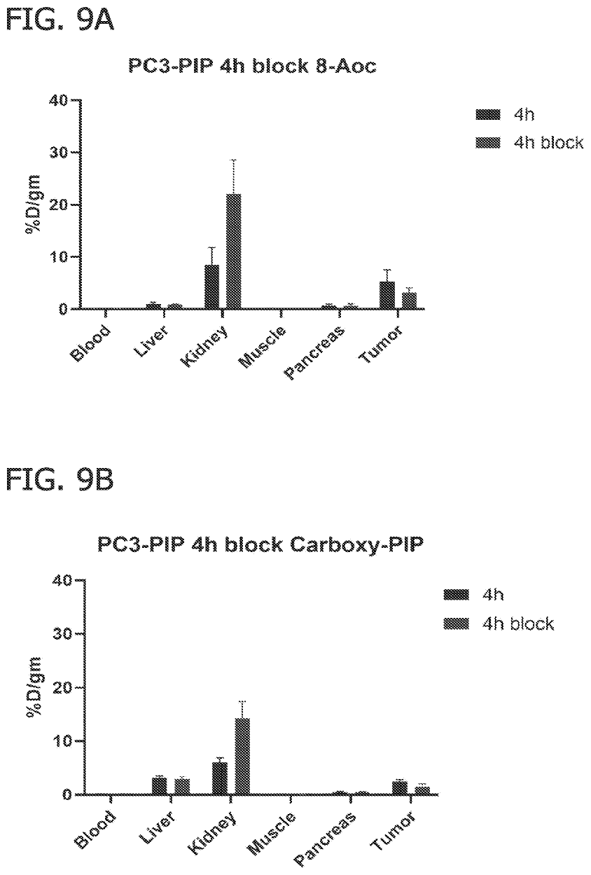

In vivo biodistribution studies were completed in male CB-17 SCID mice (n=4, 4-5 weeks old) which were xenografted with either PC3 or PC3-PIP tumors. These studies were conducted in compliance with the standards outlined in the NIH Guide for Care and Use of Laboratory Animals (8^{th} edition) and were approved by the Institutional Animal Care and Use Committee members at the American Association for Laboratory Animal Science (Protocol Number 23101, 08/2021). For biodistribution and pharmacokinetic studies, mice were injected with purified radiotracer compound (≈10 μCi, 0.37 MBq, 43 Ci/pmol) in 150 μL of isotonic saline via the tail vein. For animals with tumor blocking, mice were injected with either 100 μg of commercially available Bombesin (1-14) or 2-(Phosphonomethyl)-pentanedioic acid (PMPA) in isotonic saline 15 mins prior to the injection of the radiolabeled compound. The mice were euthanized at different time points and the tissues and organs were weighed and counted on a Perkin Elmer Wizard 3 automatic gamma counter and the percent injected dose (% ID) and percent injected dose per gram (% ID/g) in each organ or tissue calculated (see FIGS. 7-9).

### In Vivo: Molecular Imaging in Tumor Bearing Mice

MicroPET/SPECT/CT imaging of PC-3 and PC-PIP xenografted CB-17 SCID mice were conducted at 4- and 24-hour time points post I.V. administration of ~300 μCi of radiotracer into each mouse. For blocking investigations 100 mg of BBN(1-14) peptide or 2-PMPA was administered 15 mins prior to injection of the radiotracer compound in both tumors. Mice were anesthetized with 2.5% of isoflurane at induction and 2% during imaging for maintenance. The mice were kept warm using a heated water bath on the imaging bed. Data was conducted using a Bruker Albira SI Micor-PET/CT scanner and reconstructed using Vivaquant software. Upon completion of the study, the anesthetized mice were humanely sacrificed by cervical dislocation.

### Example 2

### Synthesis

CB-TE1A1P was synthesized as known in the art. The heterodimer peptide conjugation to the two chelators was carried out using the identical procedure. Briefly, CB-TE2A or CB-TE1A1P (10 gmol) and 4 equivalents of DIPEA (7.5 μL) were mixed in DMF (1 mL) and stirred at room temperature for 15 min. To the stirred solution, sulfo-NHS (20 μmol, 4.3 mg) and EDC (10 μmol, 1.9 mg) were added, and the reaction mixture was stirred at room temperature for 4 h. Thereafter, DMF was removed under reduced pressure and the crude product was washed with acetonitrile (3×10 mL) and dichloromethane (3×10 mL) respectively to yield the desired activated CB-cyclam NHS-ester. This was then used for the peptide conjugation without any further purification. To a stirred solution of peptide (1 mg, 0.55 μmol) in DMF (0.4 mL), crude activated CB-cyclam NHS ester (TE2A or TE1A1P, 5.0 gmol) was added and the reaction stirred overnight at room temperature. This was then purified using semi-prep HPLC to yield the desired peptide-CB-cyclam TE2A or TE1A1P derivative. The formation of the conjugates (FIG. 3) was characterized by ESI-MS and the yields of the peptide conjugates were calculated by a calibration curve (Concentration vs. Absorbance) of the parent peptide at 254 nm.

### DUPA-6-Ahx-(Lys-TEJA)-8AOC-BBN-ANT (1)

Yield: 42% (485 μg)

MS (ESI, positive mode): Mass (calculated) 2121.20 m/z (observed) 2121.8; 1061.8 (M/2); 708.2 (M/3); 1415.6 (2M/3)

### DUPA-6-Ahx-(Lys-TE1P)-8AOC-BBN-ANT (2)

Yield: 20% (234 μg)

MS (ESI, positive mode): Mass (calculated) 2157.18 m/z (observed) 1079.6 (M/2); 720.2 (M/3).

### Radiolabeling

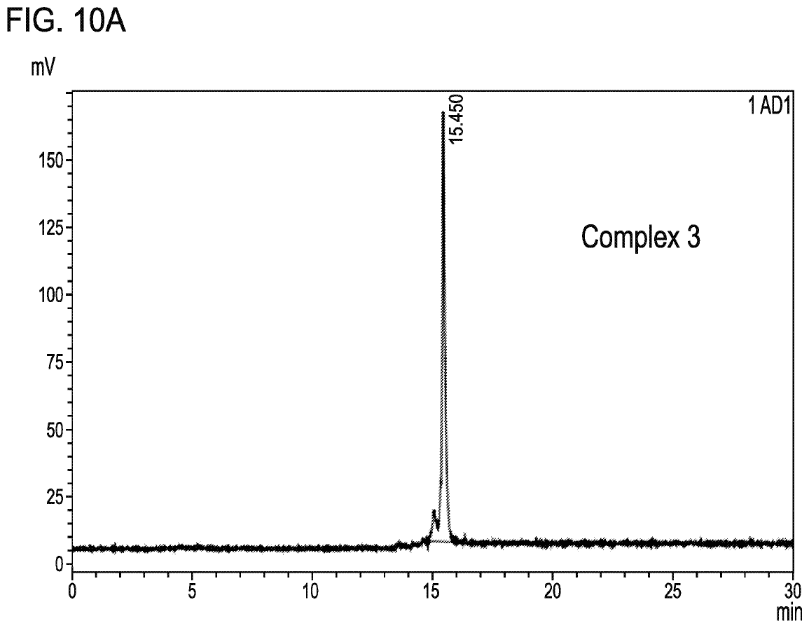

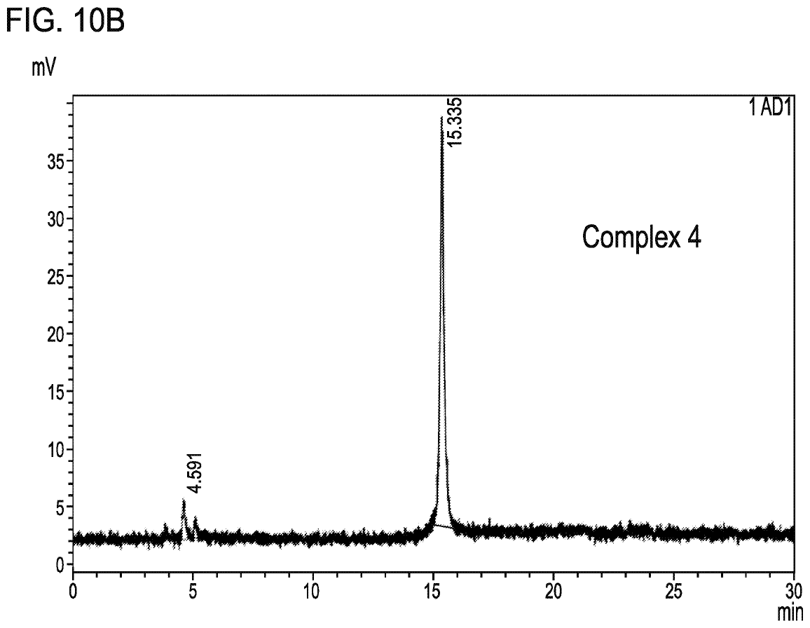

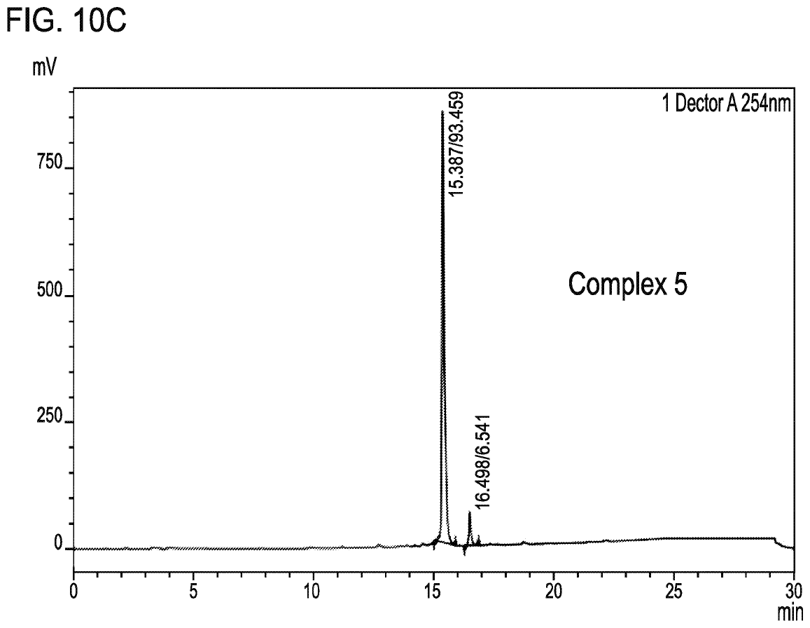

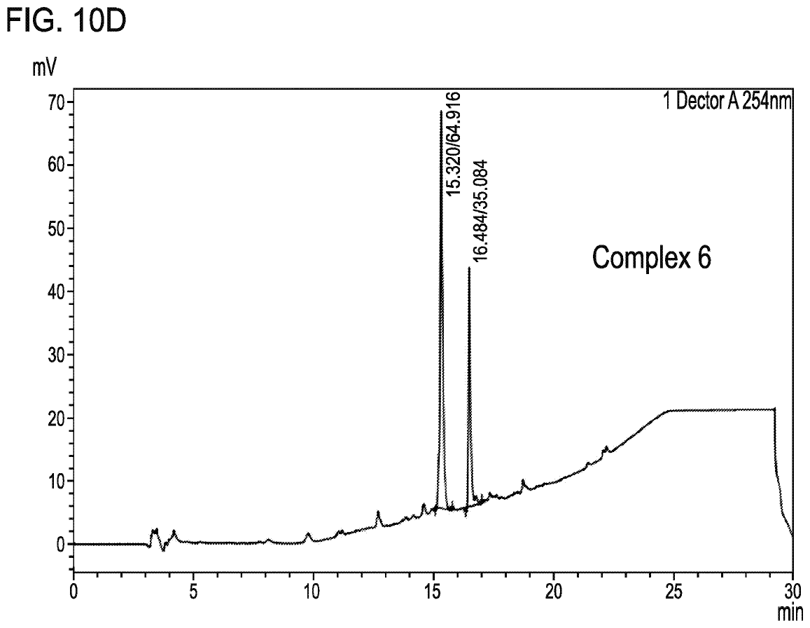

The identical labeling procedure was followed for the two peptide conjugates. ⁶⁴CuCl₂ (74 MBq; 3-5 μL) was added to the peptide conjugate (1 or 2; 8 nmol) in 0.5 M NH₄OAc (pH 5; 80 μL) and heated at 90 °C for 45 min. The formation of the complexes (3 and 4) was characterized by HPLC and the radiolabeling yields (FIGS. 10A & 10B) observed were >95%. The identity of complexes was established by preparing the natural Cu chelates of the respective peptide conjugates and characterizing them through ESI-MS.

Briefly, ^{nat}CuCl₂ (20 nmol) dissolved in water (30 μL) was added to the peptide conjugate (1 or 2; 10 nmol) in 0.5 M NH₄OAc (pH 5; 80 μL) and heated at 90 °C for 45 min. This was then characterized by UV-HPLC (at 254 nm) and ESI-MS (FIGS. 10C & 10D).

^{nat}Cu-DUPA-6-Ahx-(Lys-TE1A)-8AOC-BBN-ANT (5)

MS (ESI, positive mode): Mass (calculated) C₁₀₂H₁₅₉CuN₂₄O₂₅ 2183.12 m/z (observed) 2184.2; 1092.6 (M/2); 729.0 (M/3); 1456.4 (2M/3)

^{nat}Cu-DUPA-6-Ahx-(Lys-TE1P)-8AOC-BBN-ANT (6)

MS (ESI, negative mode): Mass (calculated) C₁₀₁H₁₅₉CuN₂₄O₂₆P 2218.09 m/z (observed) 2216.8; 1107.6 (M/2)

### Serum Stability Studies

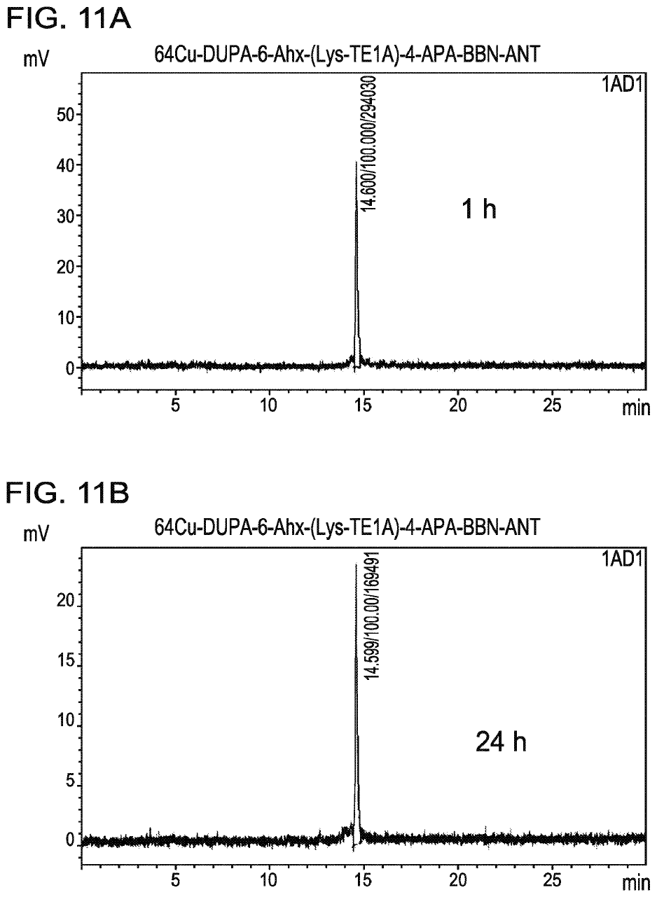

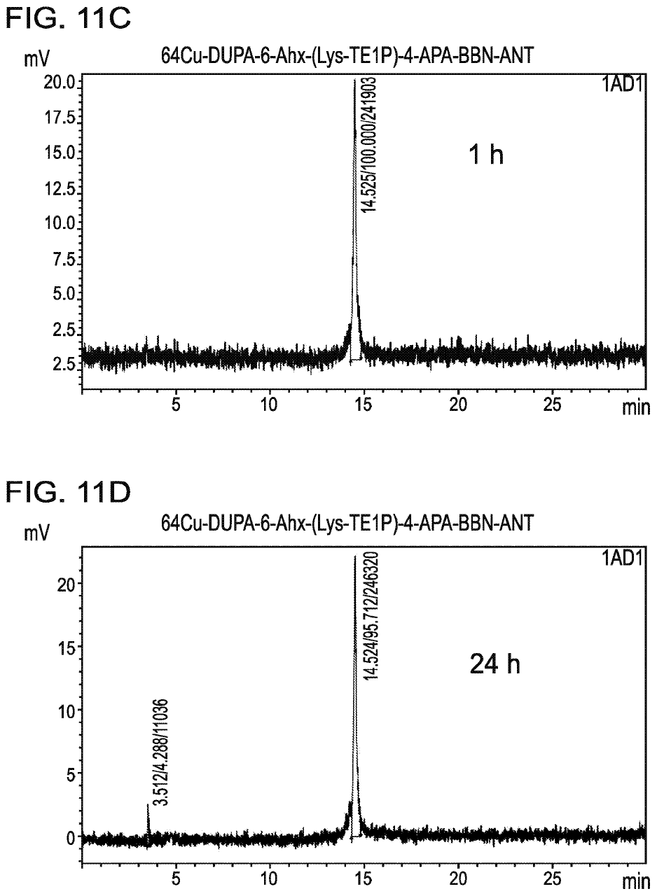

To a 450 μL of human serum, ⁶⁴Cu-complex (3 or 4; 50 μL, 37 MBq) was added and the mixture stirred at 37 °C for 1 and 4 h. At the end of each respective time point, the serum mixture (100 μL) was withdrawn, to which ethanol (500 μL) was added to precipitate out the serum proteins. The ethanolic solution was then centrifuged at 10,000 g to separate out the serum proteins and the supernatant was then analyzed by HPLC for ascertaining the serum stability over time (see FIGS. 11A-11D).

### Bio-Evaluation Studies

### Biodistribution Studies

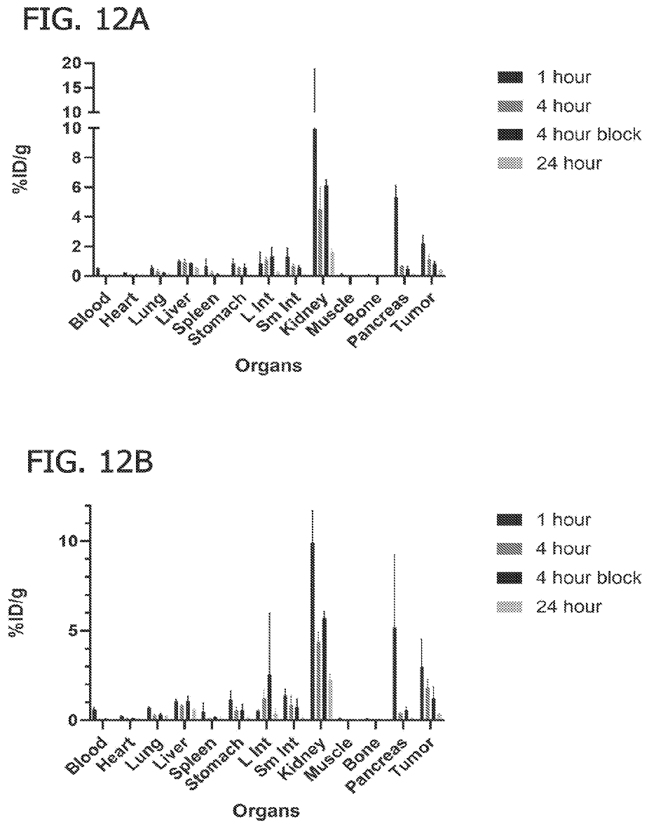

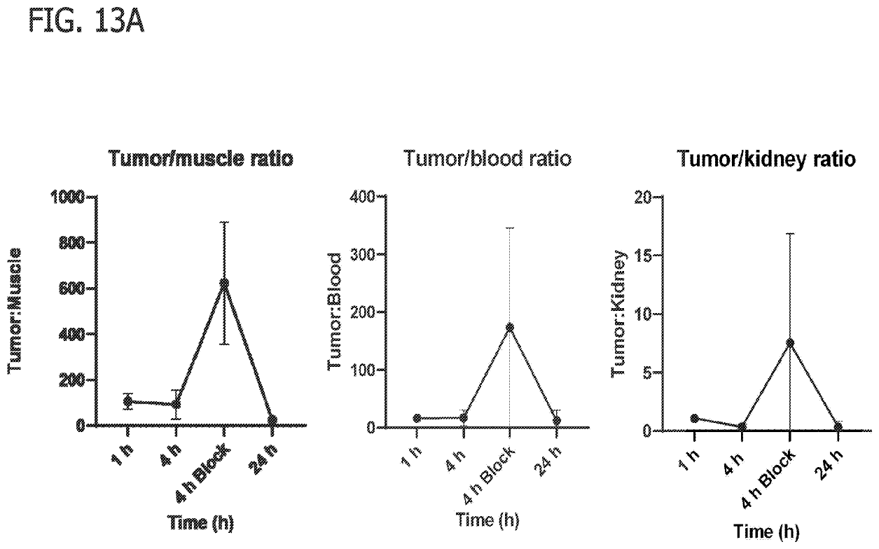

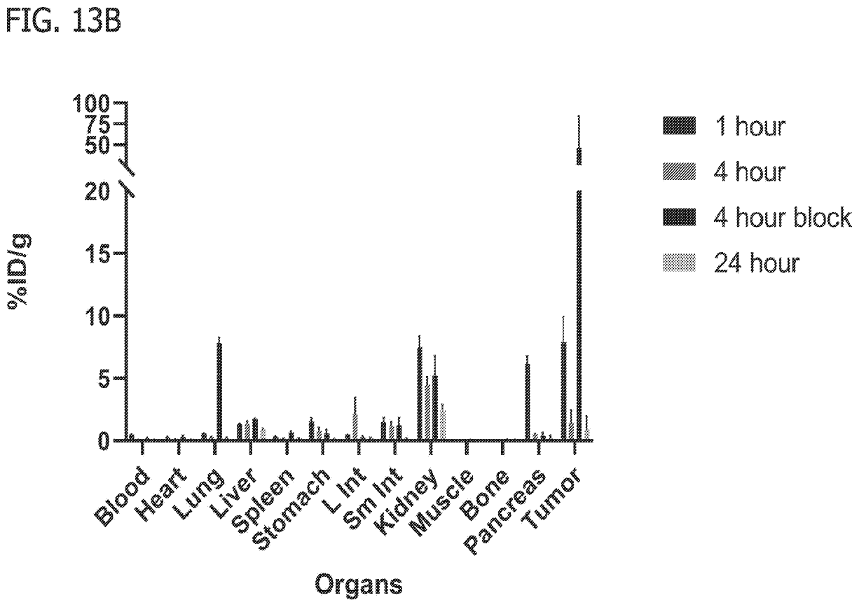

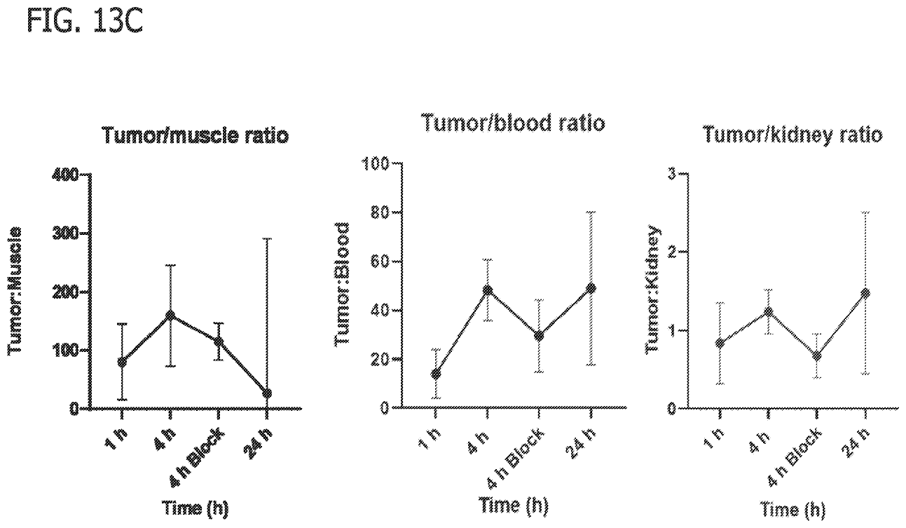

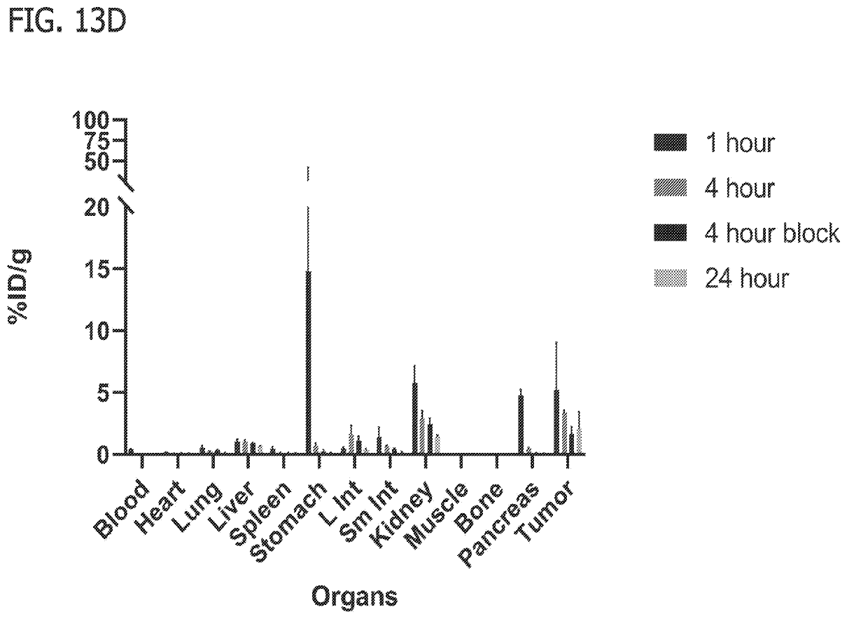

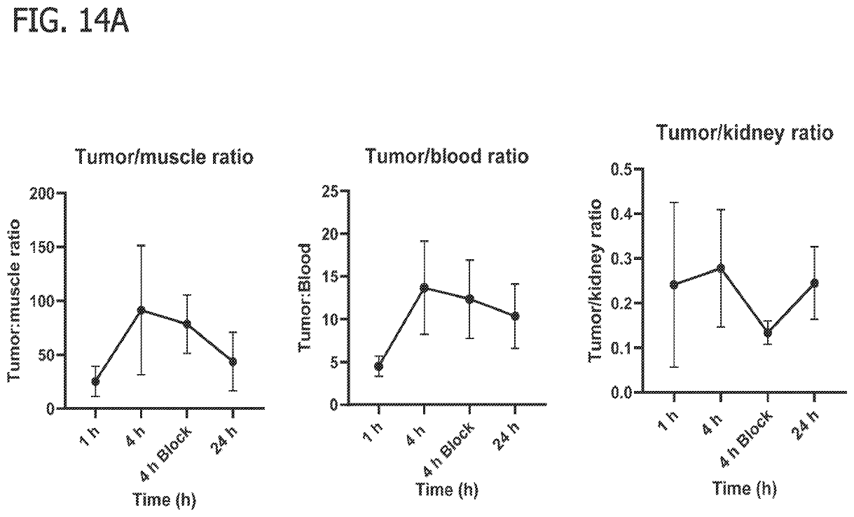

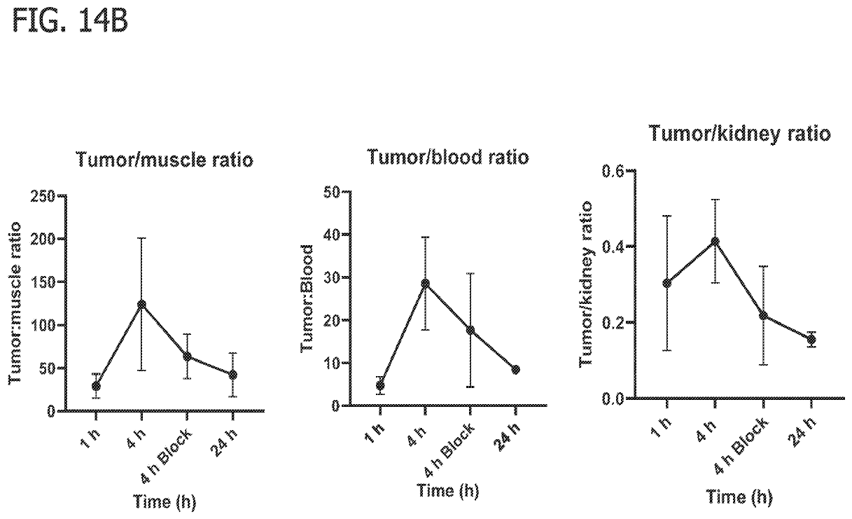

All procedures performed herein were in accordance with the national laws pertaining to the conduct of animal experiments. A solid tumor model in Nude mice bearing PC3-PIP and PC3 was used as described in Example 1. Particularly, mice were injected with purified radiotracer compound (~10 μCi, 0.37 MBq, 43 Ci/pmol) in 150 μL of isotonic saline via the tail vein. For animals with tumor blocking, mice were injected with either 100 μg of commercially available Bombesin (1-14) or 2-(Phosphonomethyl)-pentanedioic acid (PMPA) in isotonic saline 15 mins prior to the injection of the radiolabeled compound. The mice were euthanized at different time points and the tissues and organs were weighed and counted on a Perkin Elmer Wizard 3 automatic gamma counter and the percent injected dose (% ID) and percent injected dose per gram (% ID/g) in each organ or tissue calculated. Results are shown in FIGS. 12-14.

1. A radiotracer compound comprising Formula (I)
DUPA-X-M-Y-X-BBN ANT Formula (I)
wherein:
DUPA is (2-[3-(1,3-dicarboxypropyl)-ureido]pentanedioic acid);
X are independently selected from the group consisting of 4-amino-1-carboxymethly-piperidine (CP), 5-ammoniumvaleric acid (5-Ava), 6-aminohexanoic acid (6-Ahx), 8-aminooctanoic acid (8-AOC), and paraaminobenzoic acid (AMBA);
M is selected from the group consisting of [⁶⁸Ga], [¹⁷⁷Lu], [⁶⁴Cu], [⁶⁷Cu] and [¹¹¹In];
Y is a metal complexing agent selected from the group consisting of a dodecane tetraacetic acid (DOTA), [1,4,7-triazacyclododecane-1,4,7-triacetic acid](NOTA), and cross-bridged TETA phosphonic acid (CB-TE1A1P); and
BBN ANT is a BBN antagonist ligand of SEQ ID NO:1[D-Phe-Gln-Trp-Ala-Val-Gly-His-Sta-Leu-NH₂].

2. The radiotracer compound as set forth in claim 1, wherein when M is [⁶⁸Ga] or [¹⁷⁷Lu], and Y is DOTA.

3. The radiotracer compound as set forth in claim 1, wherein Y is [1,4,7,10-tetraazacyclododecane-1,4,7,10-tetraacetic acid](DO3A).

4. The radiotracer compound as set forth in claim 1, wherein when M is selected from the group consisting of [⁶⁸Ga], [⁶⁴Cu], and [⁶⁷Cu], and Y is the metal complexing agent NOTA or derivative thereof.

5. The radiotracer compound as set forth in claim 4, wherein Y is NODAGA.

6. The radiotracer compound as set forth in claim 1, wherein when M is [⁶⁴Cu] or [⁶⁷Cu], and Y is a cyclam-based (phosphonate), cross-bridged copper complexing agent CB-TE1A1P.

7. The radiotracer compound as set forth in claim 1, wherein at least one X is 6-Ahx.

8. The radiotracer compound as set forth in claim 1, wherein both X is 6-Ahx.

9. The radiotracer compound as set forth in claim 1 comprising Formula (IA)
DUPA-6-Ahx-([M]-DOTA)-X-BBN ANT Formula (IA)
wherein M is [⁶⁸Ga], [¹⁷⁷Lu] or [¹¹¹In].

10. The radiotracer compound as set forth in claim 9, wherein X is CP.

11. The radiotracer compound as set forth in claim 10 comprising formula DUPA-6-Ahx-([⁶⁸Ga]-DO3A)-X-BBN ANT.

12. The radiotracer compound as set forth in claim 10 comprising formula DUPA-6-Ahx-([¹⁷⁷Lu]-DO3A)-X-BBN ANT.

13. The radiotracer compound as set forth in claim 1 comprising Formula (IB)
DUPA-6-Ahx-([M]-NOTA)-X-BBN ANT Formula (IB)
wherein M is selected from the group consisting of [⁶⁸Ga], [⁶⁴Cu], and [⁶⁷Cu].

14. The radiotracer compound as set forth in claim 13, wherein X is CP.

15. The radiotracer compound as set forth in claim 13 selected from the group consisting of: DUPA-6Ahx-([⁶⁴Cu]Cu-NOTA)-CP-BBN ANT, DUPA-6Ahx-([⁶⁴Cu]Cu-NOTA)-8-Aoc-BBN ANT, DUPA-6Ahx-([⁶⁷Cu]Cu-NOTA)-CP-BBN ANT, DUPA-6Ahx-([⁶⁷Cu]Cu-NOTA)-8-Aoc-BBN ANT, DUPA-6Ahx-([⁶⁸Ga] Ga-NOTA)-CP-BBN ANT, and DUPA-6Ahx-([⁶⁸Ga] Ga-NOTA)-8-Aoc-BBN ANT.

16. The radiotracer compound as set forth in claim 1 comprising Formula (IC)
DUPA-6-Ahx-([M]-CB-TE1A1P)-X-BBN ANT Formula (IC)
wherein M is [⁶⁴Cu] or [⁶⁷Cu].

17. The radiotracer compound as set forth in claim 16, wherein X is CP.

18. The radiotracer compound as set forth in claim 17 selected from the group consisting of: DUPA-6Ahx-([⁶⁴Cu]Cu-CB-TE1A1P)-CP-BBN ANT, DUPA-6Ahx-([⁶⁴Cu]Cu-CB-TE1A1P)-8-Aoc-BBN ANT, DUPA-6Ahx-([⁶⁷Cu]Cu-CB-TE1A1P)-CP-BBN ANT and DUPA-6Ahx-([⁶⁷Cu]Cu-CB-TE1A1P)-8-Aoc-BBN ANT.

19. A method for detecting prostate cancer (PCa), the method comprising administering the radiotracer compound of claim 1 to a subject suspected to be at risk of PCa.

20. The method as set forth in claim 19 wherein the prostate cancer is metastatic prostate cancer.
